(19) United States
(12) Patent Application Publication
(10) Pub. No.: US 2022/0226170 A1
(43) Pub. Date: Jul. 21, 2022

(54) Thong Panty Liner for Excessive Discharge

(71) Applicant: Llona Lewis, Lansing, IL (US)

(72) Inventor: Llona Lewis, Lansing, IL (US)

(21) Appl. No.: 17/154,542

(22) Filed: Jan. 21, 2021

(51) Int. Cl. A61F 13/56; A61F 13/472

(52) U.S. Cl. CPC A61F 13/5611; A61F 13/47254

(57) ABSTRACT

A disposable feminine menstrual pad with a tail (thong-type) structured to solve the problem of leakage of overflow while horizontal or vertical. The pad is a full size menstrual pad designed to be worn with a panty, and adhered to the crotch area by a strip of adhesive. The regular size (7-9 inch long) pad of any absorbency, but usually medium to heavy flow. A tail-like portion will have an additional 7-9 inches of absorbent material that is slim enough to fit in the anal canal with comfort. The tail-like portion will not have any adhesive and will naturally adhere to the area, further secured by the friction of the buttocks cheeks.

This product addresses the issue of menstrual overflow that travels and leaks in the rear or top of the buttocks crack. This can occur on heavy flow days with blood clots or near full pads limiting space for overflow blood to travel. Or it may occur simply based on how the body is positioned, sitting or laying down and the blood travels by rolling up the anus exiting the opening area or top crack. This can be extremely embarrassing and happen with no warning. This product will eliminate this problem.

### BACKGROUND OF THE INVENTION

The present invention relates to a thong-type pad with a slim absorbent tail that fits between the user's butt cheeks to absorb heavy overflow to protect a woman during her menstrual cycle.

### DESCRIPTION OF THE PRIOR ART

The use of absorbent liners in thong-type panties to absorb discharge during a woman's menstrual cycle is known in the art. For example, U.S. Pat. No. 5,683,373 discloses a sanitary napkin shaped to fit the contours of a thong garment from the front to the crotch of the wearer. Also, U.S. Pat. No. 6,572,597 discloses a winged panty liner adapted to be used in a thong-type panty. However, neither of these prior art devices include a thong-type panty liner system having a second absorbent liner or liner portion extending rearward from the crotch portion and along the anus cavity of the wearer to absorb fluid from heavy menstrual discharges that may extend into the anus cavity.

### SUMMARY OF THE INVENTION

The present invention comprises an absorbent pad portion with a thong type pad for maximum coverage. The thong pad system is particularly adapted to absorb heavy menstrual discharge fluid that may extend rearwardly from the crotch during instances of excessive overflow discharge. The thong pad system of the present invention comprises a pad of any absorbency (for the crotch) and a slim tail-like portion of the same absorbency as the pad extending adjacent the anus cavity area, the tail-like portion extending from the rear end of the full pad and upward along the thong portion that extends along the anus cavity of the wearer.

### BRIEF DESCRIPTION OF THE DRAWINGS

The present invention will be better understood and its objectives will become apparent when consideration is given to the following detailed description of the illustrated embodiment, the latter making reference to the annexed drawings in which:

FIG. 1 is a perspective view of a pad with the absorbent tail-like portion attached to the pad of the present invention located in the crotch and anus cavity portions of the panty;

FIG. 2 is a top view of the tail-like portion attached to a full size pad;

FIG. 3 is a rear view of a wearer of the absorbent tail-like portion and regular sized pad showing the location of the heavy absorbent liner portion when the tail pad panty thong is worn.

### DETAILED DESCRIPTION OF THE ILLUSTRATED EMBODIMENT

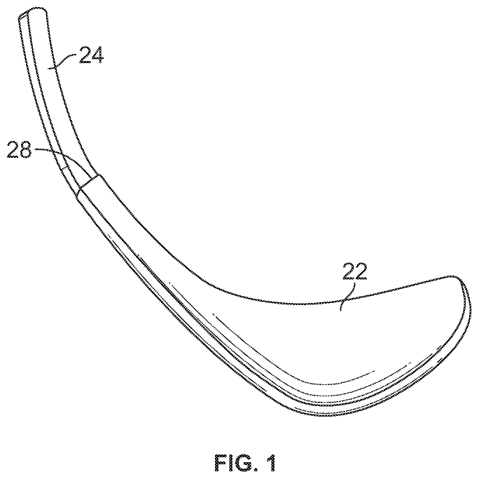

Referring to FIG. 1 panty shield 22 is used in the crotch area and is attached to the wearer's panty by adhesive, as is known in the art. Shield 22 is shaped so it does not overhang the sides of crotch portion of an average panty. Shield 22 is composed of absorbent material or materials that readily absorb a normal menstrual discharge of the wearer. Shield 22 can be held on the inner side of crotch portion and adhered to a female panty in any one of known ways, such as adhesive, hook-and-loop fasteners or the like.

Attached to the above-described shield 22 is a slim, equally or greater absorbent extension or tail-like portion of material 24 configured to line the anal cavity of the user. The tail-like portion is adapted to naturally be secured in the anal canal without the need for adhesives. Tail-like portion 24 is attached to shield 22 at 28, as shown in FIG. 1. This combination addresses the need of complete coverage for heavy fluid discharge.

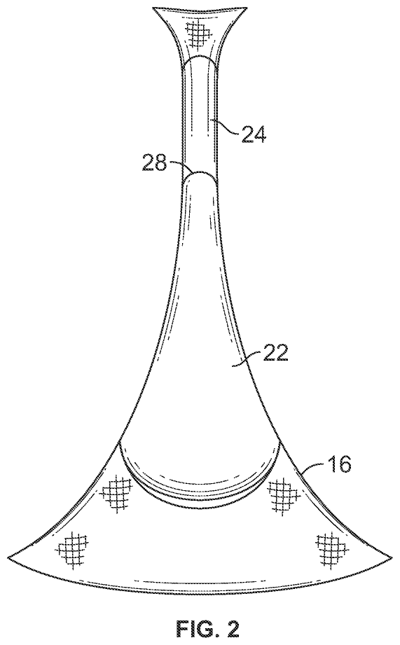

FIG. 2 is a top view illustrating the shield 22 and tail-like portion 24 lodged in the crotch and anal canal segments of panty 16, respectively. Shield 22 is adhered to the crotch portion of panty 16 by adhesive or the like. Tail-like portion 24 is attached to shield 22 at 28, but there is no adhesive or any fastener between tail-like portion 24 and panty 16.

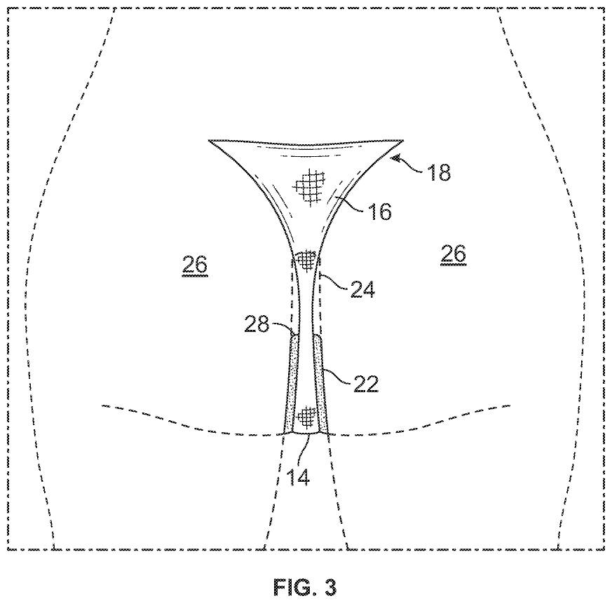

As seen in FIG. 3, when shield 22 and tail-like portion 24 are inserted into a panty 16 worn by a user, shield 22 is placed adjacent the crotch area 14 of the user. Tail-like portion 24 is maintained in position by the natural friction of the buttock cheeks 26. As seen in FIG. 3, shield 22 and tail-like portion 24 are shown slightly wider than the crotch portion 14 of panty 16. However, shield 22 and tail-like portion 24 may be the same width as crotch portion 14 and the anal canal portion of pant 16, thus eliminating overhang.

While the subject matter of the present invention has been described in one or more embodiments, it is to be understood that the specific mechanisms and structure which have been described are merely illustrative of the principles of the invention. Modifications may be made to the structure described without limiting the spirit and scope of the invention as defined by the attached claims.

I claim:

1. A combination absorbent female menstrual pad, comprising:
a first fluid-absorbent shield configured to be removably attached to a crotch segment of a female panty;
a second tail-like fluid-absorbent portion attached to and extending outwardly from the first fluid-absorbent shield, the second tail-like portion adapted to be positioned adjacent an anal canal area of a user of the female panty when the panty is worn by a female user.

2. The combination absorbent female menstrual pad of claim 1, wherein:
one surface of the first fluid-absorbent shield comprises an adhesive, the adhesive adapted to removably attach the absorbent shield to the crotch portion of the female panty.
